(19) United States
(12) Patent Application Publication
(10) Pub. No.: US 2022/0409415 A1
(43) Pub. Date: Dec. 29, 2022

(54) KNEE MOTION SUPPORT APPARATUS

(71) Applicant: Toyota Jidosha Kabushiki Kaisha, Toyota-shi Aichi-ken (JP)

(72) Inventor: Kanako Okano, Toyota-shi Aichi-ken (JP)

(73) Assignee: Toyota Jidosha Kabushiki Kaisha, Toyota-shi Aichi-ken (JP)

(21) Appl. No.: 17/846,097

(22) Filed: Jun. 22, 2022

(30) Foreign Application Priority Data
Jun. 24, 2021 (JP) 2021-104975

(51) Int. Cl. A61F 5/01; B25J 11/00

(52) U.S. Cl. CPC A61F 5/0125; B25J 11/00

(57) ABSTRACT

A knee motion support apparatus capable of adjusting, with a high degree of freedom, a resistance force that resists the force applied to a knee joint is provided. A knee motion support apparatus is attached to a leg of a user. The knee motion support apparatus includes: a pair of links rotatably connected to each other; a detection unit configured to detect an angle between the pair of links; a damper configured to apply a resistance force in a direction in which a knee joint of the leg is bent; and an adjustment unit configured to adjust the resistance force based on the detected angle between the pair of links and a change pattern of the resistance force applied by the damper.

### CROSS REFERENCE TO RELATED APPLICATIONS

This application is based upon and claims the benefit of priority from Japanese patent application No. 2021-104975, filed on Jun. 24, 2021, the disclosure of which is incorporated herein in its entirety by reference.

### BACKGROUND

The present disclosure relates to a knee motion support apparatus, and, in particular, to a knee motion support apparatus that supports a motion of a knee by applying a resistance force thereto.

An example of such a knee motion support apparatus is a motion support apparatus disclosed in Japanese Unexamined Patent Application Publication No. 2018-012148. The motion support apparatus disclosed in Japanese Unexamined Patent Application Publication No. 2018-012148 adjusts a damping force for damping a force applied to a knee joint according to whether a wearer is in a leg-standing state or a leg-swinging state.

### SUMMARY

The present inventors have found the following problem.

A user wearing a knee motion support apparatus is in various states. Although the knee motion support apparatus exerts a resistance force that resists the force applied to a knee joint, a change pattern of the resistance force is limited to one. Thus, the change pattern of the resistance force may not be suitable for a user. Therefore, there is a demand for a knee motion support apparatus capable of adjusting the resistance force with a high degree of freedom.

In view of the above-described problem, an object of the present disclosure is to provide a knee motion support apparatus capable of adjusting, with a high degree of freedom, a resistance force that resists the force applied to a knee joint.

A first exemplary aspect is a knee motion support apparatus attached to a leg of a user, the knee motion support apparatus including:

a pair of links rotatably connected to each other;

a detection unit configured to detect an angle between the pair of links;

a damper configured to apply a resistance force in a direction in which a knee joint of the leg is bent; and

an adjustment unit configured to adjust the resistance force based on the detected angle between the pair of links and a change pattern of the resistance force applied by the damper.

According to this configuration, the resistance force applied by the damper is adjusted by setting a change pattern of the resistance force for each user. Therefore, the resistance force that resists the force applied to the knee joint can be adjusted with a high degree of freedom.

Further, when a walking mode is set, the adjustment unit may determine whether a phase is a stance phase (i.e., a leg-standing phase) or a swing phase (i.e., a leg-swinging phase) based on the detected angle between the pair of links, and adjust the resistance force in accordance with the stance phase and the swing phase based on the change pattern of the resistance force, and

a magnitude of the resistance force in the stance phase may be determined in accordance with a desire of the user.

According to this configuration, by setting the magnitude of the resistance force in the stance phase for each user, the resistance force applied by the damper is adjusted. Therefore, the resistance force can be adjusted for each of the users for whom resistance forces different from each other are required in the stance phase.

Further, the adjustment unit may determine whether a phase is a stance phase or a swing phase based on the detected angle between the pair of links, and adjust the resistance force in accordance with the swing phase, a first stance phase, and a second stance phase based on the change pattern of the resistance force,

the phase may change in an order of the swing phase, the first stance phase, and the second stance phase, and

the resistance force may be increased when the phase changes from the swing phase to the first stance phase, and may be increased when the phase changes from the first stance phase to the second stance phase.

According to this configuration, the resistance force is gradually increased in the order of the swing phase, the first stance phase, and the second stance phase. Therefore, since an impact caused by a change in the resistance force is not exerted on the knee of a user in the first stance phase, the user does not feel strange. Further, in the second stance phase, a high resistance force is applied to the knee of the user, to thereby firmly support the knee of the user.

Further, the adjustment unit may determine whether a phase is a stance phase or a swing phase based on the detected angle between the pair of links, and adjust the resistance force in accordance with the swing phase, a first stance phase, and a second stance phase based on the change pattern of the resistance force,

the phase may change in an order of the swing phase, the first stance phase, and the second stance phase, and

the resistance force may be increased when the phase changes from the swing phase to the first stance phase, and may be reduced when the phase changes from the first stance phase to the second stance phase.

According to this configuration, the resistance force is increased when the phase changes from the swing phase to the first stance phase, and is reduced when the phase changes from the first stance phase to the second stance phase. Therefore, in the first stance phase, a high resistance force is applied to the knee of a user, to thereby firmly support the knee of the user. Further, since a large resistance force is not applied to the knee of a user in the second stance phase, the user does not feel strange.

Further, the adjustment unit may adjust the resistance force so that it has a predetermined value based on the change pattern of the resistance force.

According to this configuration, when a user tries to sit, the user can easily sit since a constant resistance force is applied to his/her knee.

The present disclosure can provide a knee motion support apparatus capable of adjusting, with a high degree of freedom, a resistance force that resists the force applied to a knee joint.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a perspective view showing a knee motion support apparatus according to a first embodiment;

FIG. 2 is a front view showing the knee motion support apparatus according to the first embodiment;

FIG. 3 is a side view showing the knee motion support apparatus according to the first embodiment;

FIG. 4 is a rear view showing main units of the knee motion support apparatus according to the first embodiment;

FIG. 5 is a block diagram showing a control system of the knee motion support apparatus according to the first embodiment;

FIG. 6 is a diagram showing an example of an operation performed by the knee motion support apparatus according to the first embodiment;

FIG. 7 is a diagram showing an example of an operation performed by the knee motion support apparatus according to the first embodiment;

FIG. 8 is a diagram showing an example of an operation performed by the knee motion support apparatus according to the first embodiment;

FIG. 9 is a diagram showing a state in which a user wears a knee motion support apparatus on his/her leg;

FIG. 10 is a graph showing an example of a change pattern of a resistance force;

FIG. 11 is a graph showing an example of a change pattern of a resistance force;

FIG. 12 is a graph showing an example of a change pattern of a resistance force;

FIG. 13 is a diagram showing a knee angle;

FIG. 14 is a diagram showing an upper leg angle and a lower leg angle; and

FIG. 15 is a side view showing a modified example of the knee motion support apparatus according to the first embodiment.

### DESCRIPTION OF EMBODIMENTS

A specific embodiment to which the present disclosure is applied will be described hereinafter in detail with reference to the drawings. However, the present disclosure is not limited to the following embodiment. Further, for the clarification of the description, the following descriptions and the drawings are simplified as appropriate.

### First Embodiment

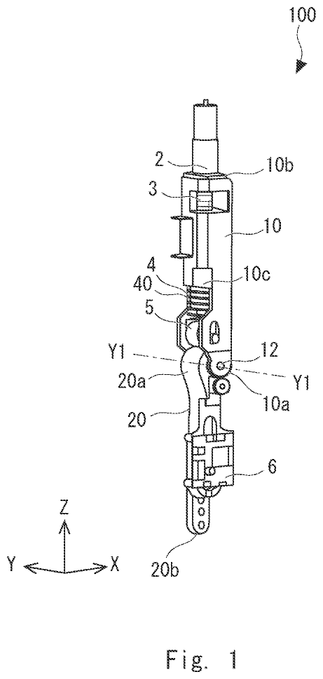

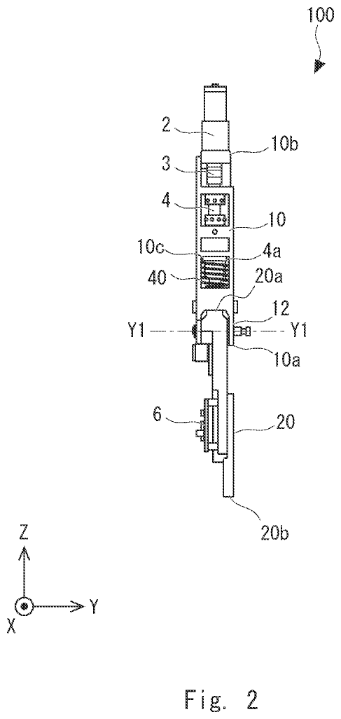

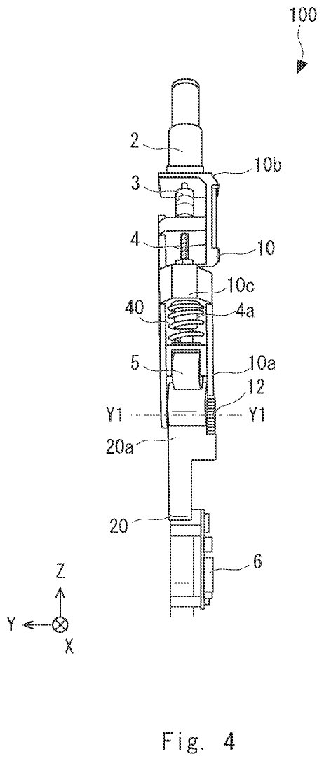

A configuration of a knee motion support apparatus according to a first embodiment will be described with reference to FIGS. 1 to 4. Note that it is needless to say that right-handed xyz orthogonal coordinates shown in FIG. 1 and other drawings are merely for convenience of describing the positional relation of the components. Normally, a z-axis positive direction is vertically upward and a xy plane is a horizontal plane, which are the same throughout the drawings.

As shown in FIG. 1, a knee motion support apparatus 100 includes an upper leg side link 10 and a lower leg side link 20. One end 10a of the upper leg side link 10 and one end 20a of the lower leg side link 20 are mechanically connected to each other so that they can rotate about a rotation axis Y1. The one end 20a of the lower leg side link 20 has a cam shape. The angle between the upper leg side link 10 and the lower leg side link 20 is, for example, 0 (zero) degrees or greater and 180 degrees or less. When the knee motion support apparatus 100 is attached to a leg of a user, the upper leg side link 10 is attached to the upper leg of the leg of the user, and the lower leg side link 20 is attached to the lower leg of the leg of the user.

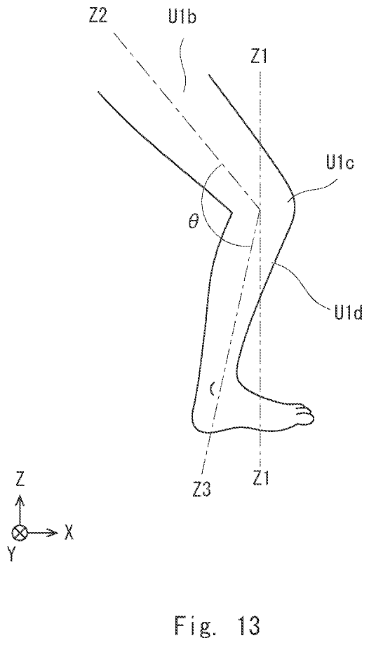

The upper leg side link 10 or the lower leg side link 20 includes a detection unit 12. The detection unit 12 is an angle sensor, and it detects an angle between the upper leg side link 10 and the lower leg side link 20. When the knee motion support apparatus 100 is attached to the leg of a user, the angle between the upper leg side link 10 and the lower leg side link 20 corresponds to a knee angle β of the user. Note that the knee angle β of the user is formed by crossing a straight line Z2 extending in the axial direction of an upper leg U1b of the user shown in FIG. 13 with a straight line Z3 extending in the axial direction of a lower leg U1d shown in FIG. 13.

The detection unit 12 outputs the angle between the upper leg side link 10 and the lower leg side link 20 to a control apparatus 6 as a knee angle detection value. The knee angle detection value indicates a waveform corresponding to a gait cycle. That is, the knee angle detection value changes periodically in accordance with the gait cycle.

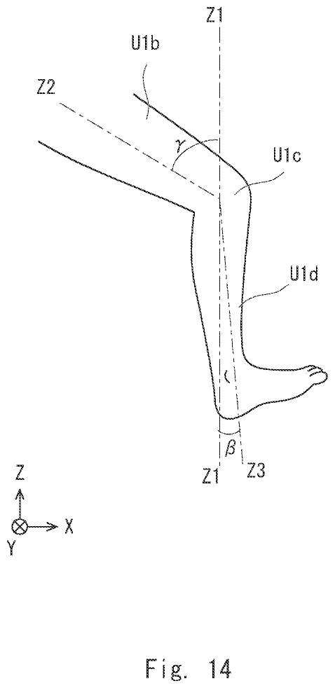

Note that the detection unit 12 may include an inertial measurement apparatus or the like in addition to the angle sensor. A lower leg angle β and an upper leg angle γ can be obtained based on the values detected by the inertia measurement apparatus and the angle sensor. As shown in FIG. 14, the lower leg angle β is formed by crossing a vertical line Z1 with the straight line Z3 extending in the axial direction of the lower leg U1d. Further, the upper leg angle γ is formed by crossing the vertical line Z1 with the straight line Z2 extending in the axial direction of the upper leg U1b. The detection unit 12 outputs the lower leg angle β and the upper leg angle γ to the control apparatus 6.

The upper leg side link 10 includes a drive unit 2, an adjustment unit 3, a damper 4, and a roller 5. The drive unit 2, the adjustment unit 3, the damper 4, and the roller 5 are held by the upper leg side link 10 in this order from an other end 10b of the upper leg side link 10 toward the one end 10a thereof.

The lower leg side link 20 includes the control apparatus 6. The control apparatus 6 according to this embodiment is provided between the one end 20a and an other end 20b of the lower leg side link 20. The control apparatus 6 is formed by hardware mainly using a microcomputer including a Central Processing Unit (CPU), a Read Only Memory (ROM), a Random Access Memory (RAM), an interface (I/F), and the like. The CPU, the ROM, the RAM, and the interface are connected to each other through a data bus or the like. The control apparatus 6 acquires a change pattern of a resistance force and a knee angle detection value of a user. Further, the control apparatus 6 acquires an angle between the upper leg side link 10 and the lower leg side link 20 from the detection unit 12.

The control apparatus 6 can detect a walking timing based on the knee angle detection value or the like detected by the detection unit 12. Specifically, the walking timing is a stance phase and a swing phase in one gait cycle.

The control apparatus 6 acquires a signal indicating a predetermined change pattern of a resistance force from a communication terminal (not shown) by using communication device such as Bluetooth (Registered Trademark) Low Energy (BLE). Further, there are various types of change patterns of the resistance force, examples of which will be described later. The control apparatus 6 generates a resistance force control signal based on the change pattern of the resistance force and the knee angle detection value. The control apparatus 6 transmits the generated resistance force control signal to the drive unit 2 by wire communication or wireless communication. Note that the control apparatus 6 may acquire the angle (the lower leg angle) between the vertical line and the lower leg side link 20 and the angle (the upper leg angle) between the vertical line and the upper leg side link 10 as appropriate.

The drive unit 2 provides rotational power to the adjustment unit 3 based on the resistance force control signal received from the control apparatus 6. The drive unit 2 includes, for example, a motor and a driver. Note that the drive unit 2 may be provided on the side of the other end 10b of the upper leg side link 10, and the drive unit 2 provides rotational power to the adjustment unit 3 via a gear or a pulley.

The adjustment unit 3 adjusts a resistance force F applied by the damper 4 based on the rotational power provided from the drive unit 2. Specifically, the adjustment unit 3 continuously changes a cross-sectional area of a flow path of a damper liquid in the damper 4 in response to receiving the rotational power of the drive unit 2.

Expression 1 expressing the relation between the resistance force F of the damper 4, a damper coefficient k, and a velocity v at which the damper liquid passes through the flow path is shown below.

F=kv (Expression 1)

Therefore, when the cross-sectional area of the flow path of the damper liquid in the damper 4 is reduced, the damper coefficient k increases, and as a result, the resistance force F applied by the damper 4 increases. By this configuration, the adjustment unit 3 adjusts the resistance force F applied by the damper 4 based on the change pattern of the resistance force F and the knee angle detection value. Note that the adjustment unit 3 may adjust the cross-sectional area of the flow path of the damper liquid in the damper 4 to a plurality of stages, and the number of stages is not limited to any particular number.

The damper 4 transmits the resistance force F to the lower leg side link 20 via the roller 5. The damper 4 includes a rod 4a and a spring 40. The rod 4a and the spring 40 are sandwiched between a damper holding part 10c of the upper leg side link 10 and the roller 5. The roller 5 is provided between the damper 4 and the one end 20a of the lower leg side link 20 so as to be rotatable and movable in the axial direction (the Z-axis direction) of the upper leg side link 10.

### (Example of Control System)

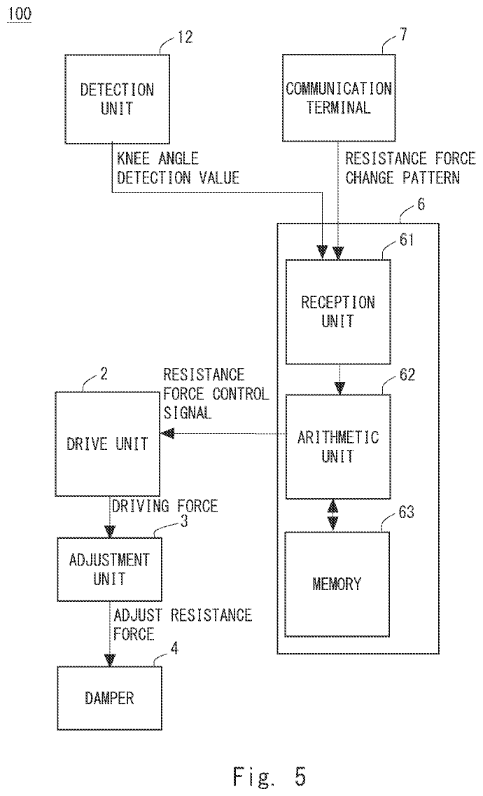

Next, an example of a control system of the knee motion support apparatus 100 will be described with reference to FIG. 5.

As shown in FIG. 5, the control apparatus 6 includes a reception unit 61, an arithmetic unit 62, and a memory 63. The arithmetic unit 62 is, for example, a CPU that performs arithmetic processing, control processing, and the like. The memory 63 is, for example, a RAM that stores various types of data or the like, in which an arithmetic program, a control program or the like executed by the arithmetic unit 62 is stored.

A communication terminal 7 presents resistance force change patterns that can be selected by a user, and receives an input indicating the resistance change pattern selected by the user. Note that the resistance force change patterns presented by the communication terminal 7 may be, for example, a walking mode and a sitting mode. The communication terminal 7 is, for example, a smartphone.

The reception unit 61 receives a knee angle detection value from the detection unit 12 and the resistance force change pattern from the communication terminal 7.

The arithmetic unit 62 generates a resistance force control signal based on the knee angle detection value and the resistance force change pattern received by the reception unit 61. The resistance force control signal indicates a change in the resistance force of the damper 4 with respect to time. The arithmetic unit 62 sends the resistance force control signal to the drive unit 2 via a predetermined interface.

The drive unit 2 acquires the resistance force control signal generated by the arithmetic unit 62 and provides a driving force to the adjustment unit 3. The adjustment unit 3 adjusts a cross-sectional area of a flow path of a damper liquid in the damper 4 based on the provided driving force. By the above, the cross-sectional area of the flow path of the damper liquid in the damper 4 is changed, and thus the resistance force applied by the damper 4 is changed.

### (Example of Operation)

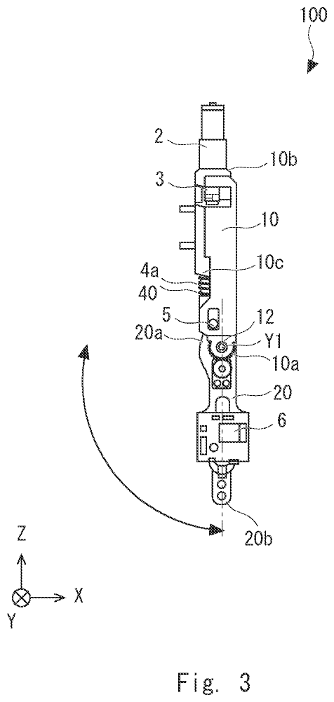

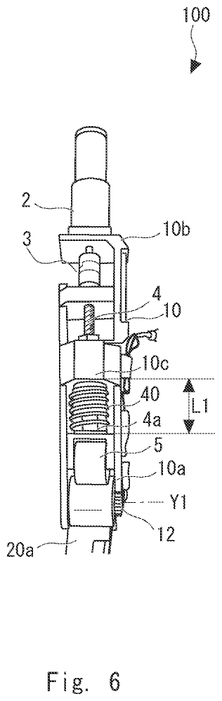

Next, an example of operations performed by the knee motion support apparatus 100 will be described with reference to FIGS. 3, 4, and 6 to 8.

As shown in FIGS. 3, 4, and 6, when the angle between the upper leg side link 10 and the lower leg side link 20 in the knee motion support apparatus 100 is approximately 180 degrees, a predetermined distance L1 is maintained between the upper leg side link 10 and the roller 5. Therefore, each of the damper 4 and the spring 40 maintains a predetermined length from the upper leg side link 10 and the roller 5 in order to receive a predetermined force. The spring 40 maintains the same length as the distance L1.

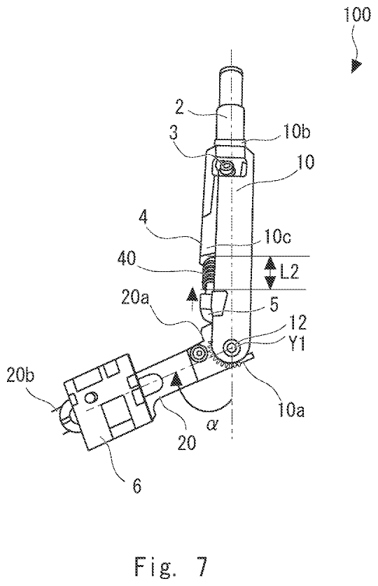

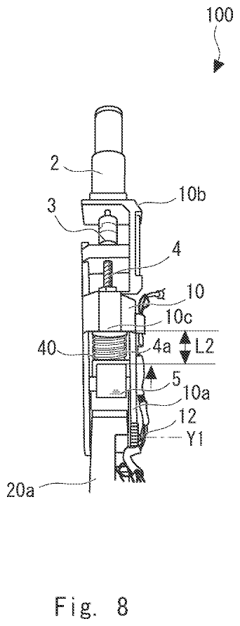

As shown in FIGS. 7 and 8, in the knee motion support apparatus 100, the lower leg side link 20 is bent at an angle α relative to the upper leg side link 10. Then, the one end 20a of the lower leg side link 20 having a cam shape pushes up the roller 5. By the above, the roller 5 approaches the upper leg side link 10, and the distance between the roller 5 and the upper leg side link 10 is reduced from the distance L1 to a distance L2. Then, the roller 5 pushes up the rod 4a and the spring 40 of the damper 4, and the damper 4 is compressed in response to receiving a force from the upper leg side link 10 and the roller 5. Therefore, the spring 40 is contracted so as to have the same length as the distance L2. Meanwhile, the lower leg side link 20 receives a reaction force from the damper 4 via the roller 5. Thus, the lower leg side link 20 receives a resistance force that resists the bending of the lower leg side link 20 by the damper 4.

### (Usage)

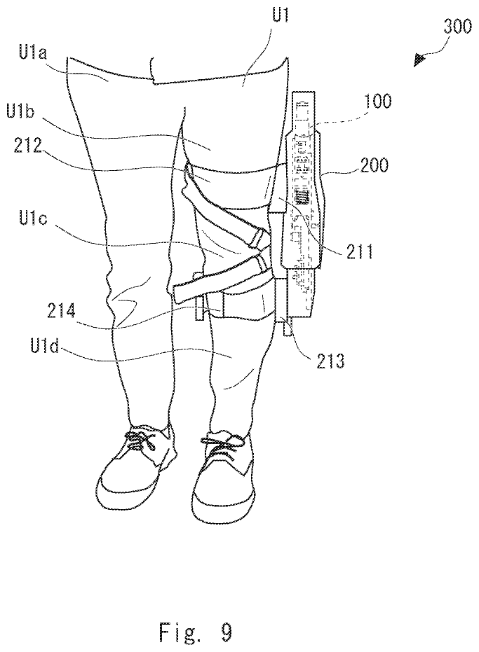

Next, a state in which a user wears a knee motion support apparatus on his/her leg will be described with reference to FIG. 9.

A knee motion support apparatus 300 shown in FIG. 9 includes, in addition to the knee motion support apparatus 100, a case 200, bases 211 and 213, and belts 212 and 214. The case 200 houses the knee motion support apparatus 100. The belt 212 is provided on an upper side surface of the case 200 via the base 211. The belt 214 is provided on a lower side surface of the case 200 via the base 213.

The upper leg U1b of a leg U1a of a user U1 is tightened using the belt 212, and the lower leg U1d of the leg U1a of the user U1 is tightened using the belt 214. Then, the knee motion support apparatus 100 can be attached to the leg U1a of the user U1. Note that a knee joint U1c of the user U1 is positioned between the belt 212 and the belt 214.

### (Change Pattern 1 of Resistance Force)

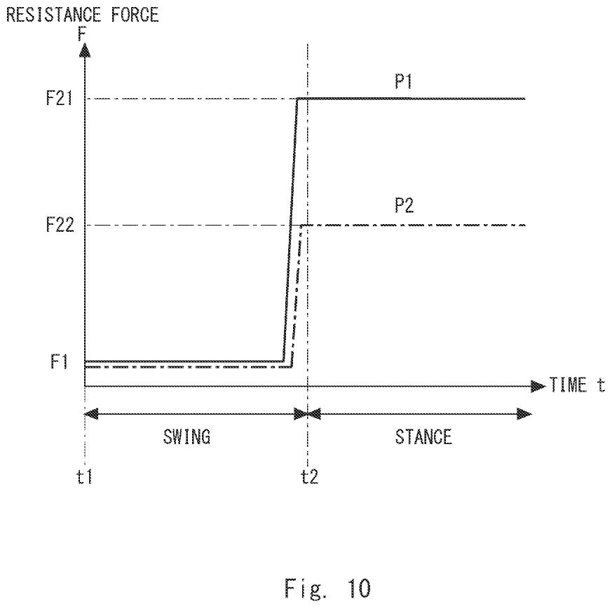

Next, an example of a change pattern of a resistance force applied by the damper 4 will be described with reference to FIG. 10.

As shown in FIG. 10, there are change patterns P1 and P2 of a resistance force (hereinafter also referred to as resistance force change patterns P1 and P2). When a user desires a high resistance force F in the stance phase of a walking motion, the user may select the resistance force change pattern P1. On the other hand, when a user desires a low resistance force F in the stance phase of a walking motion, the user may select the resistance force change pattern P2.

In the resistance force change pattern P1, a resistance force value F1 is maintained from a start time t1 of the swing phase to immediately before a start time t2 of the stance phase. Next, the resistance force is increased to a resistance force value F21 from immediately before the start time t2 of the stance phase to the start time t2 of the stance phase. Next, the resistance force value F21 is maintained after the start time t2 of the stance phase. In this way, the resistance force is changed in accordance with the swing phase and the stance phase.

In the resistance force change pattern P2, the resistance force value F1 is maintained from the start time t1 of the swing phase to immediately before the start time t2 of the stance phase. Next, the resistance force is increased to a resistance force value F22 from immediately before the start time t2 of the stance phase to the start time t2 of the stance phase. Next, the resistance force value F22 is maintained after the start time t2 of the stance phase. In this way, the resistance force is changed in accordance with the swing phase and the stance phase.

The resistance force value F21 of the resistance force change pattern P1 is larger than the resistance force value F22 of the resistance force change pattern P2. Therefore, the resistance force change pattern P1 is more suitably used by a user who desires the high resistance force F after the start time t2 of the stance phase than the resistance force change pattern P2. On the other hand, the resistance force change pattern P2 is more suitably used by a user who desires the low resistance force F after the start time t2 of the stance phase than the resistance force change pattern P1. By selecting the resistance force change pattern P1 or P2, the resistance force applied by the damper 4 can be adjusted for each user.

### (Change Pattern 2 of Resistance Force)

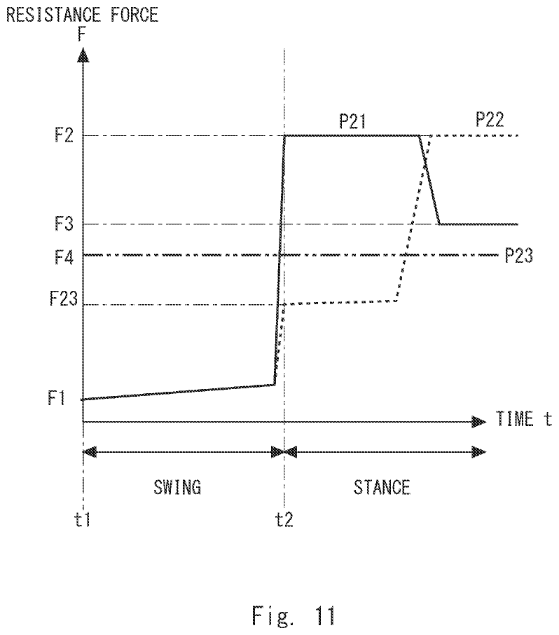

Next, another example of a change pattern of a resistance force applied by the damper 4 will be described with reference to FIG. 11.

As shown in FIG. 11, there are change patterns P21, P22, and P23 of a resistance force (hereinafter also referred to as resistance force change patterns P21, P22, and P23).

In the resistance force change pattern P21, the resistance force value F1 is maintained from the start time t1 of the swing phase to immediately before the start time t2 of the stance phase. Next, the resistance force is increased to a resistance force value F2 from immediately before the start time t2 of the stance phase to the start time t2 of the stance phase. Next, after the elapse of a predetermined period of time from the start time t2 of the stance phase, the resistance force is reduced to a resistance force value F3, and the resistance force value F3 is maintained. A period of time from the start time t2 of the stance phase until a predetermined period of time elapses may be referred to as a “first stance phase”, and a period of time from an end time of the first stance phase to an end time of the stance phase may be referred to as a “second stance phase”.

Immediately after the start time t2 of the stance phase, the knee of a user tends to be bent in the stance phase. In the resistance force change pattern P21, since the resistance force value F2 is maintained after the start time t2 of the stance phase, the damper 4 supports the knee of the user with the resistance force value F2 at a time when the knee of the user is likely to be bent. Further, since the resistance force value F3 is reduced after the elapse of a predetermined period of time from the start time t2 of the stance phase, the damper 4 supports the knee of the user with the small resistance force value F2 at a time when the knee of the user is hardly bent. By the above, it is possible to prevent a user from having a feeling of strangeness resulting from a resistance force being applied to his/her knee.

In the resistance force change pattern P22, the resistance force value F1 is maintained from the start time t1 of the swing phase to immediately before the start time t2 of the stance phase. Next, the resistance force is increased to a resistance force value F23 from immediately before the start time t2 of the stance phase to the start time t2 of the stance phase. Next, the resistance force value F23 is maintained after the start time t2 of the stance phase. Next, after the elapse of a predetermined period of time from the start time t2 of the stance phase, the resistance force is increased to the resistance force value F2, and the resistance force value F2 is maintained.

In the resistance force change pattern P22, since the resistance force value in the stance phase is gradually improved, a change in the resistance force value is small. Therefore, a user is less likely to feel a change in the resistance force value. The user can walk without much feeling that there has been a change in the resistance force value.

The resistance force change pattern P23 is used when a user selects a sitting mode. In the resistance force change pattern P23, a resistance force value F4 is maintained from the start time t1 of the swing phase to the end time of the stance phase passing through the start time t2 of the stance phase.

When the user sits, the damper 4 maintains the constant resistance force value F4 throughout the swing phase and the stance phase. By doing the above, even when the knee of the user is likely to be bent, the resistance force applied by the damper 4 supports the knee of the user. By the above, the knee of the user can be prevented from bending and thus the user can sit easily. Therefore, the resistance force change pattern P23 is suitably used when a user sits.

### (Change Pattern 3 of Resistance Force)

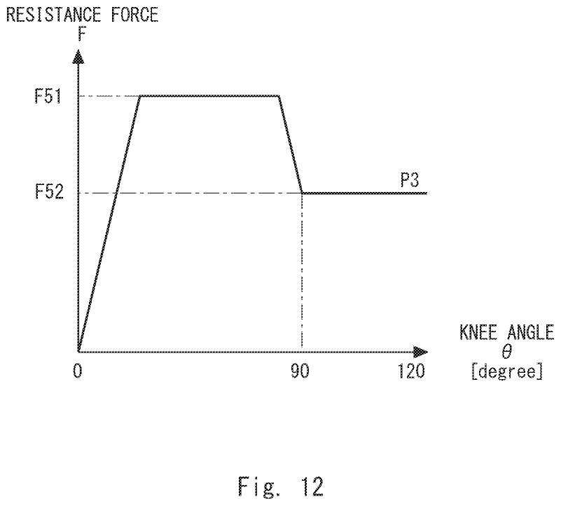

Next, still another example of a change pattern of a resistance force applied by the damper 4 will be described with reference to FIG. 12.

In a change pattern P3 of a resistance force (hereinafter also referred to as a resistance force change pattern P3) shown in FIG. 12, the resistance force changes when the knee angle β (see FIG. 13) becomes a predetermined angle or greater. Specifically, when the knee angle β increases from 0 (zero) degrees, the resistance force F is increased to a resistance force value F51, and then the resistance force value F51 is maintained until the knee angle β reaches approximately 90 degrees. Further, when the knee angle β reaches 90 degrees, the resistance force F is reduced from the resistance force value F51 to a resistance force value F52. Note that, although 90 degrees is set as a threshold in the resistance force change pattern P3 shown in FIG. 12, the threshold may be selected from a wide range of angles.

When a user walks normally, the knee angle β is often 90 or greater and less than 180 degrees. In such a case, the resistance force F applied by the damper 4 supports the knee of the user with the resistance force value F52. Further, when an abnormality occurs in the walking of the user, for example, his/her knee may be bent and hence the knee angle β may be reduced to less than 90 degrees. In such a case, when the knee angle β becomes less than 90 degrees, the resistance force F applied by the damper 4 is increased to the resistance force value F51. Thus, the knee of the user can be supported with a large resistance force so that the knee angle β becomes 90 degrees or greater. Therefore, in some embodiments, the resistance force change pattern P3 is used, because it can maintain the knee angle β at not less than 90 degrees even when an abnormality occurs in the walking of the user.

Note that the present disclosure is not limited to the above-described embodiment and may be changed as appropriate without departing from the spirit of the present disclosure. Further, the present disclosure may be implemented by combining the above-described embodiment and the example thereof as appropriate.

For example, in the above-described first embodiment, although the upper leg side link 10 includes the drive unit 2, the adjustment unit 3, the damper 4, and the roller 5 and the lower leg side link 20 includes the control apparatus 6, the upper leg side link 10 may include only at least one of the drive unit 2, the adjustment unit 3, the damper 4, the roller 5, and the control apparatus 6, and the lower leg side link 20 may include the remainder thereof.

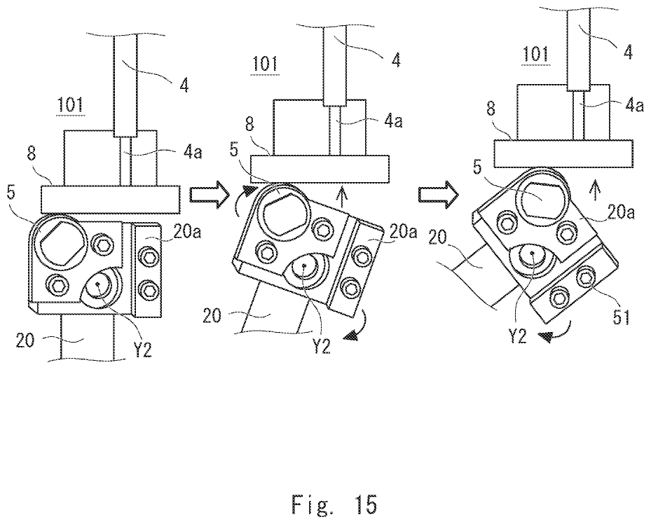

Further, as shown in FIGS. 7 and 8, in the knee motion support apparatus 100, although the upper leg side link 10 includes the roller 5, the lower leg side link 20 may include the roller 5. A knee motion support apparatus 101 shown in FIG. 15 is a modified example of the knee motion support apparatus 100 shown in FIG. 1. The lower leg side link 20 of the knee motion support apparatus 101 includes the roller 5. The roller 5 is rotatably provided at the one end 20a of the lower leg side link 20. The lower leg side link 20 is rotated clockwise as viewed from the front of the paper (i.e., the figure) around a rotation axis Y2. Then, the roller 5 pushes up the rod 4a of the damper 4 via a block 8. The resistance force applied by the damper 4 is exerted, to thereby give resistance to the rotation of the lower leg side link 20. Like in the case of the knee motion support apparatus 100, the knee motion support apparatus 101 provides a resistance force applied by the damper 4.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A knee motion support apparatus attached to a leg of a user, the knee motion support apparatus comprising:
a pair of links rotatably connected to each other;
a detection unit configured to detect an angle between the pair of links;
a damper configured to apply a resistance force in a direction in which a knee joint of the leg is bent; and
an adjustment unit configured to adjust the resistance force based on the detected angle between the pair of links and a change pattern of the resistance force applied by the damper.

2. The knee motion support apparatus according to claim 1, wherein
when a walking mode is set, the adjustment unit determines whether a phase is a stance phase or a swing phase based on the detected angle between the pair of links, and adjusts the resistance force in accordance with the stance phase and the swing phase based on the change pattern of the resistance force, and
a magnitude of the resistance force in the stance phase is determined in accordance with a desire of the user.

3. The knee motion support apparatus according to claim 1, wherein
the adjustment unit determines whether a phase is a stance phase or a swing phase based on the detected angle between the pair of links, and adjusts the resistance force in accordance with the swing phase, a first stance phase, and a second stance phase based on the change pattern of the resistance force,
the phase changes in an order of the swing phase, the first stance phase, and the second stance phase, and
the resistance force is increased when the phase changes from the swing phase to the first stance phase, and is increased when the phase changes from the first stance phase to the second stance phase.

4. The knee motion support apparatus according to claim 1, wherein
the adjustment unit determines whether a phase is a stance phase or a swing phase based on the detected angle between the pair of links, and adjusts the resistance force in accordance with the swing phase, a first stance phase, and a second stance phase based on the change pattern of the resistance force,
the phase changes in an order of the swing phase, the first stance phase, and the second stance phase, and
the resistance force is increased when the phase changes from the swing phase to the first stance phase, and is reduced when the phase changes from the first stance phase to the second stance phase.

5. The knee motion support apparatus according to claim 1, wherein the adjustment unit adjusts the resistance force so that it has a predetermined value based on the change pattern of the resistance force.
